(19) United States
(12) Patent Application Publication
(10) Pub. No.: US 2015/0336993 A1
(43) Pub. Date: Nov. 26, 2015

(54) MITOCHONDRIA-TARGETED THERANOSTIC AGENTS

(71) Applicant: The Board of Trustees of the Leland Stanford Junior University, Palo Alto, CA (US)

(72) Inventor: Zhen Cheng, Mountain View, CA (US)

(21) Appl. No.: 14/720,786

(22) Filed: May 24, 2015

Related U.S. Application Data
(60) Provisional application No. 62/003,023, filed on May 26, 2014.

(51) Int. Cl. C07F 9/6558; A61K 51/04; A61B 5/00; G01N 33/50; G01N 21/64; A61K 31/675; A61K 45/06

(52) U.S. Cl. CPC C07F 9/65583; A61K 31/675; A61K 51/0489; A61K 45/06; G01N 33/502; G01N 33/5011; G01N 21/6428; A61B 5/0071; G01N 2021/6439

(57) ABSTRACT

Mitochondria-targeted theranostic agents and methods of using them diagnostically and therapeutically are disclosed. In particular, the invention relates to theranostic agents comprising F16, or analogues thereof, conjugated to alkyltriphenylphosphonium lipophilic cations, and their uses in medical imaging and treatment of diseases associated with mitochondrial dysfunction, including cancer.

### CROSS-REFERENCE TO RELATED APPLICATION

This application claims benefit under 35 U.S.C. §119(e) of provisional application 62/003,023, filed May 26, 2014, which application is hereby incorporated by reference in its entirety.

### STATEMENT REGARDING FEDERALLY SPONSORED RESEARCH OR DEVELOPMENT

This invention was made with Government support under contract SC0008397 awarded by the Department of Energy. The Government has certain rights in this invention.

### TECHNICAL FIELD

The present invention pertains generally to mitochondria-targeted theranostic agents and methods of using them diagnostically and therapeutically. In particular, the invention relates to theranostic agents comprising F16 conjugated to alkyltriphenylphosphonium lipophilic cations and their uses in medical imaging and treatment of diseases associated with mitochondrial dysfunction.

### BACKGROUND

Mitochondria play significant roles in a variety of biological processes from cell life to death (Apostolova et al. (2011) Curr. Pharm. Des. 17:4047-4060). Mitochondria dysfunction is extensively involved in many types of human diseases (Tabrizi et al. (2000) Ann. Neurol. 47:80-86; Gogvadze (2011) Curr. Pharm. Des. 17:4034-4046) and thus has prompted research into mitochondria-specific diagnosis and therapies (Rotem et al. (2005) Cancer Res. 65:1984-1993).

Tetraphenylphosphonium and its analogue alkyltriphenylphosphonium (TPP) salts are lipophilic cations, which are able to cross the mitochondrial membrane and accumulate within the mitochondrial matrix, driven by the high membrane potential (Chen (1988) Annu Rev. Cell Biol. 4:155-181). TPP analogues have been extensively used as mitochondria targeted carriers for biomedical applications by conjugating TPP and drugs covalently (Prime et al. (2009) Proc. Natl. Acad. Sci. U.S.A. 106: 10764-10769). TPP analogues also play a significant role in mitochondria targeted imaging of diseases. For example, radionuclides such as ³H, ¹⁸F, ⁶⁴Cu, and ^{99m}Tc labelled TPP and its analogues, as well as fluorophore modified analogues, have been successfully used to study mitochondria-related events in cellular and animal models (Min et al. (2004) J. Nucl. Med. 45:636-643; Cheng et al. (2005) J. Nucl. Med. 46:878-886; Gurm et al. (2012) JACC Cardiovasc Imaging 5:285-292; Kim et al. (2012) Bioconjug. Chem. 23:431-437; Madar et al. (2007) Eur. J. Nucl. Med. Mol. Imaging 34:205720-205765; Wang et al. (2007) J. Med. Chem. 50:5057-5069; Kim et al. (2008) J. Med. Chem. 51:2971-2984; Chalmers et al. (2012) J. Am. Chem. Soc. 134:758-761; Cocheme et al. (2012) Nat. Protoc. 7:946-958).

The demand for new therapeutics targeting mitochondria has prompted the discovery of new agents that interfere with the physiological activities of mitochondria. A small molecule, 4-[(E)-2-(indol-3-yl)ethenyl]-N-methylpyridinium iodide (F16), is an exemplary agent having both fluorescent imaging and therapeutic properties, and has been found to be useful in treating cancer (Fantin et al. (2002) Cancer Cell 2:29-42; Fantin et al. (2004) Cancer Res. 64:329-336). As a delocalized cationic (DLC) compound, F16 exhibits excellent optical properties with fluorescent emission in the visible region. F16 also shows mitochondria-specific accumulation in a variety of cancer cells where it is cytotoxic due to its ability to trigger apoptosis and necrosis of cancer cells (Fantin et al. (2002), supra; Fantin et al. (2004), supra). The integration of diagnostic and therapeutic capabilities in F16 makes it useful as a theranostic agent.

There remains a need for better methods of diagnosing, monitoring, and treating diseases associated with mitochondrial dysfunction. The discovery of novel, improved theranostic agents will allow targeted therapy to be combined with medical imaging and should find use in numerous applications, including monitoring the localization and therapeutic efficacy of therapeutic agents, cell mitochondrial imaging, and image-guided surgery.

### SUMMARY

The invention relates to mitochondria-targeted theranostic agents and methods of using them diagnostically and therapeutically. In particular, the invention relates to theranostic agents comprising F16, or analogues thereof, conjugated to alkyltriphenylphosphonium lipophilic cations and their uses in medical imaging and treatment of diseases and conditions associated with mitochondrial dysfunction, including cancer.

Mitochondria-targeted theranostic agents that can be used in the practice of the invention include compounds comprising TPP conjugated to F16, or various analogues thereof, having the chemical formula:

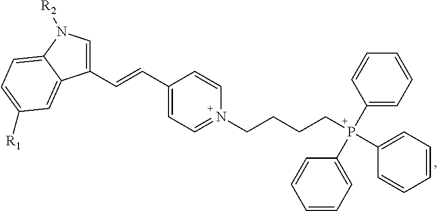

or a pharmaceutically acceptable salt thereof, wherein R₁ is a hydrogen atom, a halogen atom, an alkyl group, or an aryl group, and R₂ is a hydrogen atom or an alkyl group.

In one embodiment, the mitochondria-targeted theranostic agent comprises a MeF16-TPP conjugate having the formula:

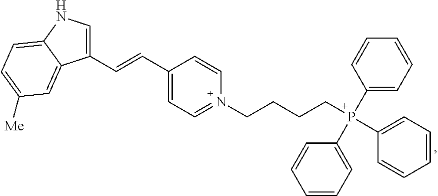

or a pharmaceutically acceptable salt thereof.

In another embodiment, the mitochondria-targeted theranostic agent comprises a FF16-TPP conjugate having the formula:

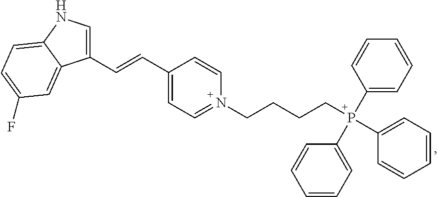

or a pharmaceutically acceptable salt thereof.

In certain embodiments, the invention includes a composition comprising at least one mitochondria-targeted theranostic agent and a pharmaceutically acceptable excipient. In one embodiment, the composition further comprises one or more other drugs for treating a disease or condition. For example, the composition may further comprise one or more chemotherapeutic agents.

Mitochondria-targeted theranostic agents have fluorescence and cytotoxicity in addition to mitochondrial targeting characteristics. In particular, mitochondria-targeted theranostic agents have the ability to cause apoptosis and decrease cell proliferation of a target cell containing dysfunctional mitochondria. Therefore, mitochondria-targeted theranostic agents can be used to treat diseases and disorders associated with mitochondrial dysfunction.

Diseases and disorders associated with mitochondrial dysfunction include mitochondrial cytopathies that are caused by mutations, acquired or inherited, in mitochondrial DNA (mtDNA) or in nuclear genes that code for mitochondrial components, as well as any disease or condition associated with mitochondrial dysfunction that results from acquired mitochondrial dysfunction, such as caused by the adverse effects of drugs, diseases, infections, or other environmental causes. Such diseases and disorders associated with mitochondrial dysfunction include, but are not limited to mitochondrial diseases, such as mtDNA depletion, mitochondrial myopathy, diabetes mellitus and deafness, Leber's hereditary optic neuropathy, Wolff-Parkinson-White syndrome, multiple sclerosis-type disease, Leigh syndrome, neuropathy, ataxia, retinitis pigmentosa, and ptosis, myoneurogenic gastrointestinal encephalopathy, myoclonic epilepsy with ragged red fibers, mitochondrial myopathy, encephalomyopathy, lactic acidosis, stroke-like symptoms (MELAS, and mitochondrial neurogastrointestinal encephalomyopathy), as well as diseases that lead to mitochondrial dysfunction, such as, but not limited to cancer, cardiovascular diseases, liver diseases, degenerative diseases or disorders, autoimmune disorders, aging, HIV infection, Parkinson's disease, diabetes, Friedreich's ataxia, and myopathies caused by oxidative stress or DNA mutation.

In one embodiment, the invention includes a method for treating a subject for a disease or disorder associated with mitochondrial dysfunction, the method comprising administering to a subject a therapeutically effective amount of a composition comprising a mitochondria-targeted theranostic agent, wherein the mitochondria-targeted theranostic agent causes apoptosis and decreases cell proliferation of target cells in the subject that uptake the mitochondria-targeted theranostic agent into mitochondria. The method may further comprise monitoring uptake of the mitochondria-targeted theranostic agent by mitochondria in cells of the subject by detecting fluorescence from the mitochondria-targeted theranostic agent. In one embodiment, the method further comprises recording a fluorescence image of cells that uptake the mitochondria-targeted theranostic agent into mitochondria of the subject.

The mitochondria-targeted theranostic agent may be administered by any suitable mode of administration. In certain embodiments, the mitochondria-targeted theranostic agent is administered intravenously, intra-arterially, subcutaneously, or intralesionally to the subject. In one embodiment, the mitochondria-targeted theranostic agent is administered locally into a tumor of the subject.

In another embodiment, the invention includes a method for treating cancer comprising administering to a subject in need thereof a therapeutically effective amount of a composition comprising a mitochondria-targeted theranostic agent. In one embodiment, the cancer is breast cancer. In another embodiment, the cancer is glioma. In certain embodiments, the method further comprises administering a therapeutically effective amount of a chemotherapeutic agent. Multiple cycles of treatment may be administered to the subject for a time period sufficient to effect at least a partial tumor response or, more preferably, a complete tumor response.

Fluorescence emitted from the F16 (or analogue) portion of conjugates can be used for fluorescence labeling of mitochondria and in vivo imaging of cells and tissues that uptake the conjugate into mitochondria. Fluorescence may be monitored by any suitable method. For example, fluorescence of mitochondria-targeted theranostic agents can be detected by a fluorimeter, a fluorescence microscope, a fluorescence microplate reader, a fluorometric imaging plate reader, fluorescence-activated cell sorting, a fiber-optic fluorescence imaging system, or a medical fluorescence imaging device (e.g., a handheld fluorescence microscope, laparoscope, endoscope, or microendoscope).

Additionally, fuorescence images may be recorded by any suitable method. For example, a charge-coupled device (CCD) image sensor, a CMOS image sensor, or a digital camera may be used to capture images. The image may be a still photo or a video in any format (e.g., bitmap, Graphics Interchange Format, JPEG file interchange format, TIFF, or mpeg). Alternatively, images may be captured by an analog camera and converted into an electronic form. Fluorescence imaging of cells and tissues may be useful in various fields of medicine, including but not limited to oncology, neurology, and cardiology.

In one embodiment, the invention includes a method of using a mitochondria-targeted theranostic agent for monitoring mitochondria in a cell, the method comprising:

a) contacting the cell with the mitochondria-targeted theranostic agent, wherein mitochondria of the cell uptake the mitochondria-targeted theranostic agent;
b) illuminating the cell with light at a fluorescence excitation wavelength of the mitochondria-targeted theranostic agent; and c) detecting fluorescence emitted by the mitochondria-targeted theranostic agent.

In another embodiment, the invention includes a method of using a mitochondria-targeted theranostic agent for fluorescence imaging of a cell, the method comprising:

a) contacting the cell with the mitochondria-targeted theranostic agent, wherein mitochondria of the cell uptake the mitochondria-targeted theranostic agent;
b) illuminating the cell with light at a fluorescence excitation wavelength of the mitochondria-targeted theranostic agent; and c) recording a fluorescence image of the cell by detecting fluorescence emitted by the mitochondria-targeted theranostic agent.

Fluorescence emitted from a mitochondria-targeted theranostic agent can be used to monitor uptake of a mitochondria-targeted theranostic agent by mitochondria in cells of a subject. In one embodiment, the method comprises recording a fluorescence image of cells that uptake a mitochondria-targeted theranostic agent into mitochondria of a subject, such as cancerous cells or cells of a tumor. In one embodiment, the method further comprises monitoring anti-tumor activity of the mitochondria-targeted theranostic agent by recording one or more fluorescence images of cells of a tumor after uptake of the mitochondria-targeted theranostic agent into mitochondria of the subject.

In another embodiment, the invention includes a method of simultaneously treating and imaging a tumor, the method comprising: a) contacting the tumor with a mitochondria-targeted theranostic agent, wherein mitochondria in cells of the tumor uptake the compound, thereby causing apoptosis and decreasing cell proliferation of the cells of the tumor; b) illuminating the tumor with light at a fluorescence excitation wavelength of the mitochondria-targeted theranostic agent; and c) detecting fluorescence emitted by the mitochondria-targeted theranostic agent from mitochondria in the cells of the tumor.

In another embodiment, the invention includes a method of performing fluorescence image-guided surgery on a subject, the method comprising:

a) contacting mitochondria in a tissue of interest with a mitochondria-targeted theranostic agent, wherein the mitochondria uptake the mitochondria-targeted theranostic agent;
b) illuminating the tissue of interest with light at a fluorescence excitation wavelength of the mitochondria-targeted theranostic agent; c) recording a fluorescence image by detecting fluorescence emitted by the mitochondria-targeted theranostic agent with a fluorescence imaging device; and d) performing surgery on the subject. In one embodiment, the fluorescence imaging device is a medical fluorescence imaging device (e.g., a handheld fluorescence microscope, laparoscope, endoscope, or microendoscope). In certain embodiments, the fluorescence imaging device is a miniaturized fluorescence imaging system. Fluorescence may be used, for example, for detection of pathology, evaluation of the completeness of resection, visualization of critical structures, or evaluation of the efficacy of treatment.

In another embodiment, the invention includes a method for monitoring the efficacy of a therapy for treating cancer in a subject, the method comprising administering a mitochondria-targeted theranostic agent, as described herein, to the subject, whereby mitochondria in cancerous cells of the subject uptake the mitochondria-targeted theranostic agent; illuminating the cancerous cells with light at a fluorescence excitation wavelength of the mitochondria-targeted theranostic agent; and fluorescence imaging the cancerous cells in vivo in the subject after the subject undergoes the therapy, wherein fluorescence emitted from the mitochondria-targeted theranostic agent in mitochondria of the cancerous cells is detected.

In another aspect, the invention includes a method of making a mitochondria-targeted theranostic agent, the method comprising: a) reacting a 1,4-dimethylpyridinium salt in the presence of catalytic amounts of piperidine with an indole compound having the formula:

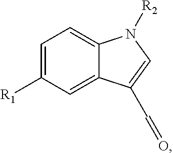

wherein R₁ is a hydrogen atom, a halogen atom, an alkyl group, or an aryl group, and R₂ is a hydrogen atom or an alkyl group, to produce a first reaction intermediate; b) reacting 4-picoline with a (4-bromobutyl)triphenylphosphonium salt to produce 4-picoline-alkyltriphenylphosphonium as the second reaction intermediate; and c) reacting the first reaction intermediate with the second reaction intermediate in the presence of catalytic amounts of piperidine to produce a mitochondrial-targeted theranostic agent, as described herein. In certain embodiments, the indole compound is selected from the group consisting of indole-3-carboxaldehyde, 5-fluro-indole-3-carboxaldehyde, and 5-methyl-indole-3-carboxaldehyde.

In yet another aspect, the invention provides a kit comprising a composition containing at least one mitochondria-targeted theranostic agent. The composition included in the kit may further comprise a pharmaceutically acceptable excipient. The kit may also include one or more additional drugs or chemotherapeutic agents. Additionally, the kit may further contain means for administering a mitochondria-targeted theranostic agent to a subject. The kit may also include instructions for use of a mitochondria-targeted theranostic agent in diagnosing, treating, or monitoring a disease or disorder associated with mitochondrial dysfunction. For example, the kit may include instructions for diagnosing and treating cancer or monitoring cancer progression in a subject.

These and other embodiments of the subject invention will readily occur to those of skill in the art in view of the disclosure herein.

### BRIEF DESCRIPTION OF THE FIGURES

FIG. 1 shows an illustration of functions of F16-TPP conjugates.

FIG. 2 shows the synthesis of the F16 and TPP derivatives.

FIG. 3A shows absorption (left, thin line) and fluorescence (FL) spectra (right, thick line) of F16 and F16-TPP related probes (5 μM) in PBS buffer (pH=7.4). From the top to the bottom: MeF16-TPP. FF16-TPP; FF16; F16 and F16-TPP. FIG. 3B shows the uptake of the F16 related probes in U87MG cells. From the left to right column are brightfield, MitoTrack, probe and overlay in each group, respectively. The FL intensity range for F16-TPP and FF16-TPP is 1000-2000, while for other probes is 2000-4000.

FIG. 4 shows the relative uptake of all F16 and F16-TPP analogues. The data was obtained by calculating by the following equation: Fluorescence signal in cell lines/Fluorescence signal in PBS buffer and normalized to F16 in the U87MG cell line. n=9. Data are presented as mean±SD.

FIGS. 5A and 5B show the antiproliferative effect on the F16 and F16-TPP analogues. Data are expressed in cell proliferative ratio with exposure of the compounds for 4 days to the negative control in PBS buffer. FIG. 5A shows cells that were treated with 5 μM compounds. FIG. 5B shows cells that were treated with 10 μM compounds. n=4. Data are presented as mean±SD.

FIGS. 6A-6E show the antiproliferative effects of the F16 and F16-TPP analogues. Data are shown for F16 (FIG. 6A), FF16 (FIG. 6B), F16-TPP (FIG. 6C), FF16-TPP (FIG. 6D), and MeF16-TPP (FIG. 6E). Data are expressed in cell proliferative ratio with exposure of the compounds for 4 days to the negative control in PBS buffer. The proliferation status of treated cultures was determined by direct counting of cells. Data are presented as mean±SD (n=4).

FIG. 7 shows fluorescence studies of both U87MG and NIH 3T3 cells, which were treated with F16. From the left to right columns are differential interference contrast (DIC) images, probes, and overlay channels in each group, respectively.

FIG. 8 shows fluorescence studies of both U87MG and NIH 3T3 cells that were treated with FF16. From the left to right column are DIC images, probes, and overlay channels in each group, respectively.

FIG. 9 shows fluorescence studies of both U87MG and NIH 3T3 cells that were treated with F16-TPP. From the left to right column are DIC images, probes, and overlay channels in each group, respectively.

FIG. 10 shows fluorescence studies of both U87MG and NIH 3T3 cells that were treated with FF16-TPP. From the left to right column are DIC images, probes, and overlay channels in each group, respectively.

FIG. 11 shows fluorescence studies of both U87MG and NIH 3T3 cells that were treated with MeF16-TPP. From the left to right column are DIC images, probes, and overlay channels in each group, respectively.

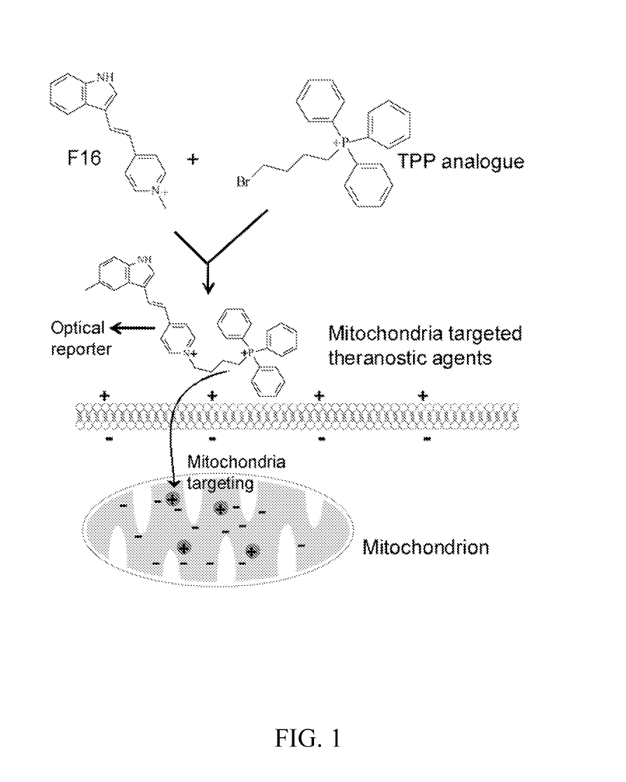

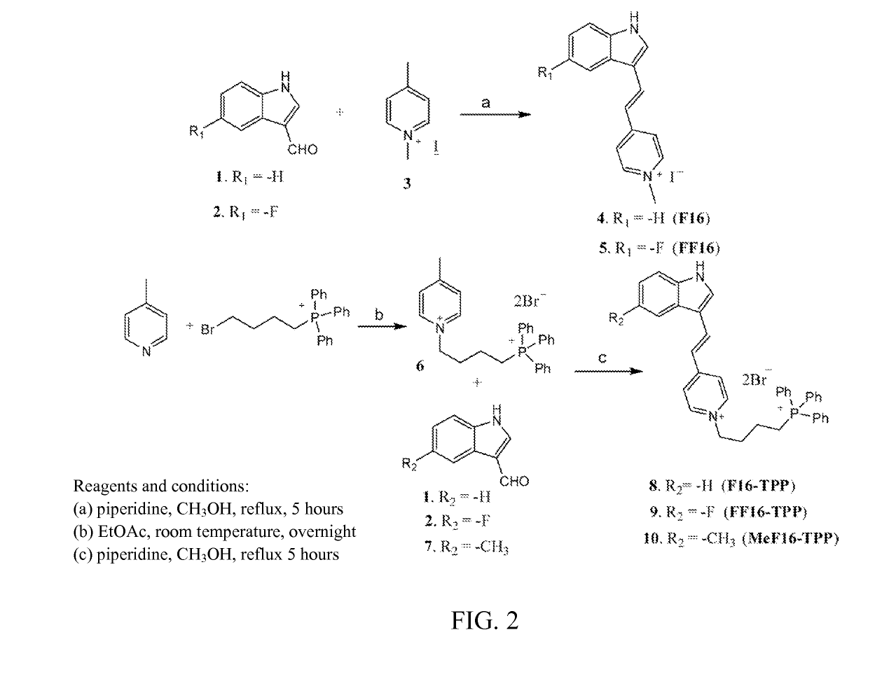

### DETAILED DESCRIPTION

The practice of the present invention will employ, unless otherwise indicated, conventional methods of medicine, pharmacology, chemistry, and biochemistry, within the skill of the art. Such techniques are explained fully in the literature. See, e.g., Cancer Theranostics (X. Chen and S. Wong eds. Academic Press, 2014); Targeted Molecular Imaging (Imaging in Medical Diagnosis and Therapy series, M. J. Welch and W. C. Eckelman eds., CRC Press, 2012); A. L. Lehninger, Biochemistry (Worth Publishers, Inc., current addition); Sambrook, et al., Molecular Cloning: A Laboratory Manual (3^{rd} Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a cell” includes a mixture of two or more cells, and the like.

The term “about,” particularly in reference to a given quantity, is meant to encompass deviations of plus or minus five percent.

The term “mitochondria-targeted theranostic agent,” as used herein, refers to a compound having the formula:

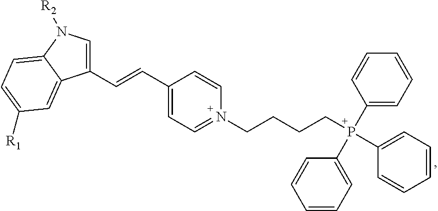

wherein R₁ is a hydrogen atom, a halogen atom, an alkyl group, or an aryl group, and R₂ is a hydrogen atom or an alkyl group. In addition, a mitochondria-targeted theranostic agent has fluorescence characteristics and localizes to mitochondria of cells where it triggers apoptosis and reduces cell proliferation.

The term “fluorescence characteristics” means an ability to emit fluorescence by irradiation of excitation light. The fluorescence characteristics of a mitochondria-targeted theranostic agent may be comparable to or different from those of 4-[(E)-2-(Indol-3-yl)ethenyl]-N-methylpyridinium iodide (F16). Examples of parameters of the fluorescence characteristics include fluorescence intensity, excitation wavelength, fluorescence wavelength, and pH sensitivity.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Diseases and disorders associated with mitochondrial dysfunction” include mitochondrial cytopathies that are caused by mutations, acquired or inherited, in mitochondrial DNA (mtDNA) or in nuclear genes that code for mitochondrial components as well as any disease or condition associated with mitochondrial dysfunction that results from acquired mitochondrial dysfunction, such as caused by the adverse effects of drugs, diseases, infections, or other environmental causes. Such diseases and disorders associated with mitochondrial dysfunction include, but are not limited to mitochondrial diseases, such as mtDNA depletion, mitochondrial myopathy, diabetes mellitus and deafness, Leber's hereditary optic neuropathy, Wolff-Parkinson-White syndrome, multiple sclerosis-type disease, Leigh syndrome, neuropathy, ataxia, retinitis pigmentosa, and ptosis, myoneurogenic gastrointestinal encephalopathy, myoclonic epilepsy with ragged red fibers, mitochondrial myopathy, encephalomyopathy, lactic acidosis, stroke-like symptoms (MELAS, and mitochondrial neurogastrointestinal encephalomyopathy), as well as diseases that lead to mitochondrial dysfunction, such as, but not limited to cancer, cardiovascular diseases, liver diseases, degenerative diseases or disorders, autoimmune disorders, aging, HIV infections, Parkinson's disease, diabetes, Friedreich's ataxia, and myopathies caused by oxidative stress or DNA mutation.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma. These terms include, but are not limited to, breast cancer, prostate cancer, lung cancer, ovarian cancer, testicular cancer, colon cancer, pancreatic cancer, gastric cancer, hepatic cancer, leukemia, lymphoma, adrenal cancer, thyroid cancer, pituitary cancer, renal cancer, brain cancer, skin cancer, head cancer, neck cancer, oral cavity cancer, tongue cancer, and throat cancer.

An “effective amount” of a mitochondria-targeted theranostic agent is an amount sufficient to effect beneficial or desired results, such as an amount that triggers apoptosis or reduces cell proliferation of cells that uptake the agent into mitochondria. An effective amount can be administered in one or more administrations, applications, or dosages.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

By “therapeutically effective dose or amount” of a mitochondria-targeted theranostic agent is intended an amount that, when administered as described herein, brings about a positive therapeutic response with respect to treatment of an individual for a disease or disorder associated with mitochondrial dysfunction, such as an amount that triggers apoptosis or reduces cell proliferation of cells that have dysfunctional mitochondria. For example, in the treatment of cancer, a therapeutically effective dose or amount is an amount having anti-tumor activity. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions.

“Substantially purified” generally refers to isolation of a substance (e.g., compound, molecule, agent) such that the substance comprises the majority percent of the sample in which it resides. Typically in a sample, a substantially purified component comprises 50%, preferably 80%-85%, more preferably 90-95% of the sample.

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; primates, and transgenic animals.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of mitochondrial targeted theranostic agents that are useful in diagnostic and therapeutic applications for treating cancer and other diseases and conditions associated with mitochondrial dysfunction. In particular, an F16-TPP conjugate and various analogues (FF16-TPP and MeF16-TPP) were synthesized and shown to target mitochondria and inhibit cancer cell growth (see Example 1). Coupling F16, or analogues thereof, with TPP produces conjugates having both optical imaging and cytotoxic properties that can be used for fluorescent imaging and treatment of cells containing dysfunctional mitochondria. In addition, such conjugates may find applications in mitochondrial imaging and image guided surgery.

In order to further an understanding of the invention, a more detailed discussion is provided below regarding the identified mitochondria-targeted theranostic agents and their diagnostic and therapeutic uses for cancer and other diseases and disorders associated with mitochondrial dysfunction.

A. Mitochondria-Targeted Theranostic Agents

Mitochondria-targeted theranostic agents that can be used in the practice of the invention include compounds comprising TPP conjugated to F16, or various analogues thereof, having the chemical formula:

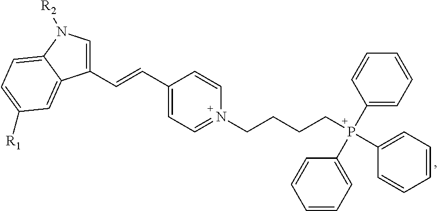

or a pharmaceutically acceptable salt thereof, wherein R₁ is a hydrogen atom, a halogen atom, an alkyl group, or an aryl group, and R₂ is a hydrogen atom or an alkyl group.

In one embodiment, the mitochondria-targeted theranostic agent comprises a MeF16-TPP conjugate having the formula:

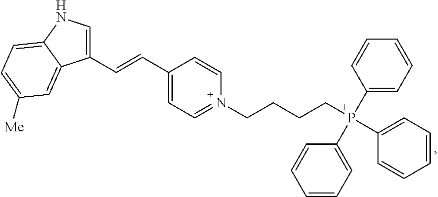

or a pharmaceutically acceptable salt thereof.

In another embodiment, the mitochondria-targeted theranostic agent comprises a FF16-TPP conjugate having the formula:

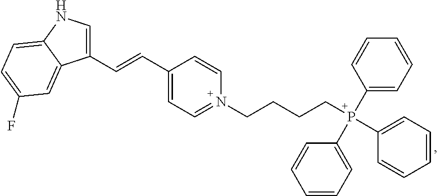

or a pharmaceutically acceptable salt thereof.

Such mitochondria-targeted theranostic agents have fluorescence, cytotoxicity, and mitochondrial targeting characteristics. In particular, mitochondria-targeted theranostic agents have the ability to cause apoptosis and decrease cell proliferation of a target cell containing dysfunctional mitochondria. For example, mitochondria-targeted theranostic agents can be used to treat tumors and cancerous cells and have anti-tumor activity. Fluorescence emitted from the F16 (or analogue) portion of conjugates can be used for in vivo imaging of cells that uptake the conjugate into mitochondria.

B. Pharmaceutical Compositions

Mitochondria-targeted theranostic agents (e.g., F16-TPP, FF16-TPP, and MeF16-TPP) can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the mitochondria-targeted theranostic agent, or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the mitochondria-targeted theranostic agent (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising one or more mitochondria-targeted theranostic agents (e.g., F16-TPP, FF16-TPP, and MeF16-TPP) described herein are in unit dosage form, meaning an amount of a conjugate or composition of the invention appropriate for a single dose, in a premeasured or pre-packaged form.

The compositions herein may optionally include one or more additional agents, such as other drugs for treating cancer or other diseases or disorders associated with mitochondrial dysfunction, or other medications used to treat a subject for a condition or disease. Particularly preferred are compounded preparations including a at least one mitochondria-targeted theranostic agent (e.g., F16-TPP, FF16-TPP, and MeF16-TPP) and one or more drugs for treating cancer or other diseases or disorders associated with mitochondrial dysfunction, such as other chemotherapeutic agents, including, but not limited to, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon. Alternatively, such agents can be contained in a separate composition from the composition comprising a mitochondria-targeted theranostic agent (e.g., F16-TPP, FF16-TPP, and MeF16-TPP) and co-administered concurrently, before, or after the composition comprising a mitochondria-targeted theranostic agent of the invention.

C. Administration

At least one therapeutically effective cycle of treatment with a mitochondria-targeted theranostic agent (e.g., F16-TPP, FF16-TPP, and MeF16-TPP) will be administered to a subject for treatment of a disease or disorder associated with mitochondrial dysfunction. Diseases and disorders associated with mitochondrial dysfunction include mitochondrial cytopathies that are caused by mutations, acquired or inherited, in mitochondrial DNA (mtDNA) or in nuclear genes that code for mitochondrial components. Mitochondrial dysfunction may also be the result of acquired mitochondrial dysfunction due to adverse effects of drugs, diseases, infections, or other environmental causes. Such diseases and disorders associated with mitochondrial dysfunction include, but are not limited to mitochondrial diseases, such as mtDNA depletion, mitochondrial myopathy, diabetes mellitus and deafness, Leber's hereditary optic neuropathy, Wolff-Parkinson-White syndrome, multiple sclerosis-type disease, Leigh syndrome, neuropathy, ataxia, retinitis pigmentosa, and ptosis, myoneurogenic gastrointestinal encephalopathy, myoclonic epilepsy with ragged red fibers, mitochondrial myopathy, encephalomyopathy, lactic acidosis, stroke-like symptoms (MELAS, and mitochondrial neurogastrointestinal encephalomyopathy), as well as diseases that lead to mitochondrial dysfunction, such as, but not limited to cancer, cardiovascular diseases, liver diseases, degenerative diseases or disorders, autoimmune disorders, aging, HIV infection, Parkinson's disease, diabetes, Friedreich's ataxia, and myopathies caused by oxidative stress or DNA mutation.

By “therapeutically effective cycle of treatment” is intended a cycle of treatment that when administered, brings about a positive therapeutic response with respect to treatment of an individual for a disease or disorder associated with mitochondrial dysfunction. Of particular interest is a cycle of treatment with a mitochondria-targeted theranostic agent that triggers apoptosis or reduces cell proliferation of cells that have dysfunctional mitochondria. For example, a cycle of treatment may have anti-tumor activity. By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy.

In certain embodiments, multiple therapeutically effective doses of compositions comprising one or more mitochondria-targeted theranostic agents (e.g., F16-TPP, FF16-TPP, and MeF16-TPP), and/or one or more other therapeutic agents, such as other chemotherapeutic drugs, or other medications will be administered. The compositions of the present invention are typically, although not necessarily, administered orally, via injection (subcutaneously, intravenously, or intramuscularly), by infusion, or locally. Additional modes of administration are also contemplated, such as intralesion, intraparenchymatous, pulmonary, rectal, transdermal, transmucosal, intrathecal, pericardial, intra-arterial, intraocular, intraperitoneal, and so forth.

The preparations according to the invention are also suitable for local treatment. In a particular embodiment, a composition of the invention is used for localized delivery of a mitochondria-targeted theranostic agent, for example, for the treatment of a tumor or cancer. For example, compositions may be administered locally into a tumor or cancerous cells of a subject. The particular preparation and appropriate method of administration are chosen to target the mitochondria-targeted theranostic agent to the site where cellular apoptosis or reduced cell proliferation is desired.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising one or more mitochondria-targeted theranostic agents and other agents may be administered using the same or different routes of administration in accordance with any medically acceptable method known in the art.

In another embodiment, the pharmaceutical compositions comprising one or more mitochondria-targeted theranostic agents and/or other agents are administered prophylactically, e.g., to prevent abnormal cell proliferation or cancer progression. Such prophylactic uses will be of particular value for subjects with a previous history of cancer or tumor growth.

In another embodiment of the invention, the pharmaceutical compositions comprising one or more mitochondria-targeted theranostic agents and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The invention also provides a method for administering a conjugate comprising a mitochondria-targeted theranostic agent as provided herein to a patient suffering from a condition that is responsive to treatment with a mitochondria-targeted theranostic agent contained in the conjugate or composition. The method comprises administering, via any of the herein described modes, a therapeutically effective amount of the conjugate or drug delivery system, preferably provided as part of a pharmaceutical composition. The method of administering may be used to treat any condition that is responsive to treatment with a mitochondria-targeted theranostic agent. More specifically, the compositions herein are effective in treating cancer and other diseases and disorders associated with mitochondrial dysfunction.

Those of ordinary skill in the art will appreciate which conditions a specific mitochondria-targeted theranostic agent can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

Generally, a therapeutically effective amount will range from about 0.50 mg to 5 grams of a mitochondria-targeted theranostic agent daily, more preferably from about 5 mg to 2 grams daily, even more preferably from about 7 mg to 1.5 grams daily. Preferably, such doses are in the range of 10-600 mg four times a day (QID), 200-500 mg QID, 25-600 mg three times a day (TID), 25-50 mg TID, 50-100 mg TID, 50-200 mg TID, 300-600 mg TID, 200-400 mg TID, 200-600 mg TID, 100 to 700 mg twice daily (BID), 100-600 mg BID, 200-500 mg BID, or 200-300 mg BID. The amount of compound administered will depend on the potency of the specific mitochondria-targeted theranostic agent and the magnitude or effect desired and the route of administration.

A purified mitochondria-targeted theranostic agent (again, preferably provided as part of a pharmaceutical preparation) can be administered alone or in combination with one or more other therapeutic agents, such as other chemotherapeutic agents, including, but not limited to, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon; or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

A mitochondria-targeted theranostic agent can be administered prior to, concurrent with, or subsequent to other agents. If provided at the same time as other agents, one or more mitochondria-targeted theranostic agents can be provided in the same or in a different composition. Thus, one or more mitochondria-targeted theranostic agents and other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising mitochondria-targeted theranostic agent and a dose of a pharmaceutical composition comprising at least one other agent, such as another mitochondria-targeted theranostic agent or drug for treating cancer or other disease or disorder associated with mitochondrial dysfunction, which in combination comprise a therapeutically effective dose, according to a particular dosing regimen. Similarly, one or more mitochondria-targeted theranostic agents and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

C. Mitochondrial Fluorescence Labeling and Imaging

Fluorescence emitted from the F16 (or analogue) portion of conjugates can be used for fluorescence labeling of mitochondria and in vivo imaging of cells and tissue that uptake the conjugate into mitochondria. Fluorescence may be monitored by any suitable method. For example, fluorescence of mitochondria-targeted theranostic agents can be detected by a fluorometer, a fluorescence microscope, a fluorescence microplate reader, a fluorometric imaging plate reader, fluorescence-activated cell sorting, a fiber-optic fluorescence imaging system, or a medical fluorescence imaging device (e.g., a handheld fluorescence microscope, laparoscope, endoscope, or microendoscope).

Additionally, fuorescence images may be recorded by any suitable method. For example, a charge-coupled device (CCD) image sensor, a CMOS image sensor, or a digital camera may be used to capture images. The image may be a still photo or a video in any format (e.g., bitmap, Graphics Interchange Format, JPEG file interchange format, TIFF, or mpeg). Alternatively, images may be captured by an analog camera and converted into an electronic form.

Fluorescence imaging with mitochondria-targeted theranostic agents, as described herein, is generally applicable for any disease, disorder, or pathology which is related to mitochondria, such as mitochondrial cytopathies, cancer, cardiovascular diseases, liver diseases, degenerative diseases or disorders, autoimmune disorders, aging, HIV infections, Parkinson's disease, diabetes, Friedreich's ataxia, myopathies caused by oxidative stress or DNA mutation, or any other diseases or disorders associated with mitochondrial dysfunction.

Preferably, a detectably effective amount of the mitochondria-targeted theranostic agent is administered to a subject; that is, an amount that is sufficient to yield an acceptable image using the fluorescence imaging equipment that is available for clinical use. A detectably effective amount of the mitochondria-targeted theranostic agent may be administered in more than one injection if needed. The detectably effective amount of the mitochondria-targeted theranostic agent needed for an individual may vary according to factors such as the degree of susceptibility to uptake into mitochondria, the age, sex, and weight of the individual, and the particular medical fluorescence imaging device used. Optimization of such factors is within the level of skill in the art.

Fluorescence imaging with mitochondria-targeted theranostic agents can be used in assessing efficacy of therapeutic drugs in treating a disease or disorder associated with mitochondrial dysfunction. For example, fluorescence images can be acquired after treatment with a mitochondria-targeted theranostic agent to determine if the individual is responding to treatment. In a subject with cancer, fluorescence imaging with a mitochondria-targeted theranostic agent can be used to evaluate whether a tumor is shrinking or growing. Further, the extent of cancerous disease (stage of cancer progression) can be determined to aid in determining prognosis and evaluating optimal strategies for treatment (e.g., surgery, radiation, or chemotherapy).

Additionally, mitochondria-targeted theranostic agents can be used in fluorescence image-guided surgery. Cells or tissues of interest can be contacted with the mitochondria-targeted theranostic agent, such that mitochondria of the cells or tissues of interest uptake the mitochondria-targeted theranostic agent. The cells or tissue are then illuminated with light at a fluorescence excitation wavelength of the mitochondria-targeted theranostic agent, and a fluorescence image of the cells or tissue is recorded using a medical fluorescence imaging device capable of detecting the fluorescence emitted by the mitochondria-targeted theranostic agent. Fluorescence imaging according to the methods of the invention can be used, for example, for detection of pathology, tumor margin delineation, evaluation of the completeness of resection, visualization of critical structures, visualization of nerves, vascular imaging, sentinel lymph node mapping, and evaluation of the efficacy of treatment.

In one embodiment, fluorescence imaging with mitochondria-targeted theranostic agents is performed with near-infrared (near-IR) light, which has the advantage that it can penetrate several millimeters to centimeters into living tissues and be used to visualize tissue below the surface. Because tissue exhibits almost no autofluorescence in the near-IR spectrum, interfering background fluorescence is minimal.

Various medical fluorescence imaging systems have been developed for open surgery as well as for laparoscopic, thoracoscopic, and robot-assisted surgery and can be used in the practice of the invention. Conventional laparoscopes and endoscopes can be equipped with an illumination source and filtered cameras to provide fluorescence guidance during medical procedures. Miniaturized fluorescence imaging systems allow imaging inside small cavities and constricted spaces. Fiber-optic fluorescence imaging systems include portable handheld microscopes, flexible endoscopes, and microendoscopes. Miniaturized fluorescence imaging devices (e.g., microendoscopes) may be implanted within a subject for long-term imaging studies. An imaging system that can simultaneously detect fluorescence at multiple wavelengths can be used for detection of fluorescence from multiple fluorescent agents that emit fluorescence at different wavelengths. In some devices, the excitation light source and photodetector are integrated into the medical device. In other devices, the excitation light source and/or photodetector reside apart and are used with remote delivery of excitation light. For a review of medical fluorescence imaging devices and methods of using them in image-guide surgery and other medical procedures, see, e.g., Gray et al. (2012) Biomed. Opt. Express. 3(8):1880-1890; Flusberg et al. (2005) Nat. Methods 2(12):941-950; Choyke et al. (2012) IEEE J. Sel. Top. Quantum. Electron. 18(3):1140-1146; Gray et al. (2012) Proc. SPIE February 3: 8207; Vahrmeijer et al. (2013) Nat. Rev. Clin. Oncol. 10:507-518; herein incorporated by reference in their entireties.

D. Kits

The invention also provides kits comprising one or more containers holding compositions comprising at least one mitochondria-targeted theranostic agent and optionally one or more other chemotherapeutic agents. Compositions can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of using the compositions comprising mitochondria-targeted theranostic agents for treating a subject for cancer or other disease or disorder involving mitochondrial dysfunction. The instructions may also describe methods of using the compositions to image cells or tissues having dysfunctional mitochondria, and methods of diagnosing and monitoring disease progression and therapeutic efficacy. The package insert can be an unapproved draft package insert or can be a package insert approved by the Food and Drug Administration (FDA) or other regulatory body.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Design, Synthesis and Biological Evaluation of Mitochondria Targeted Theranostic Agents

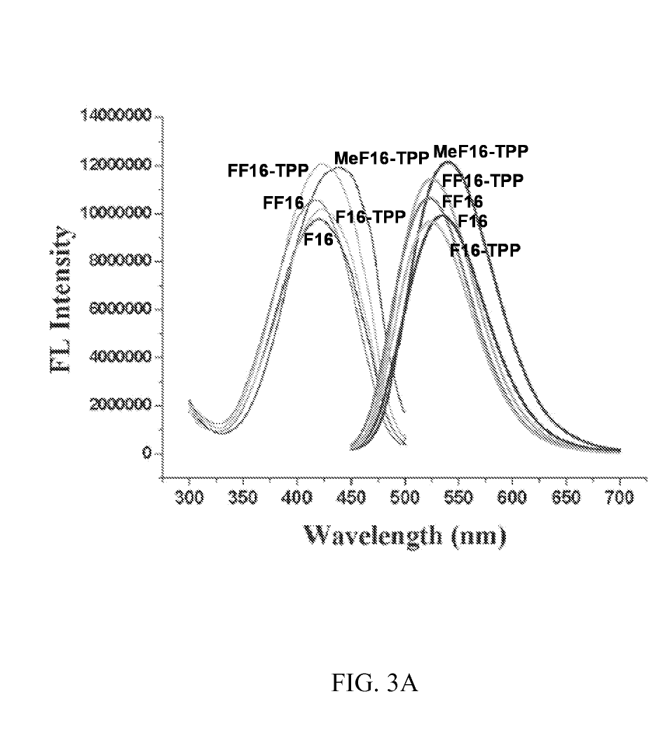

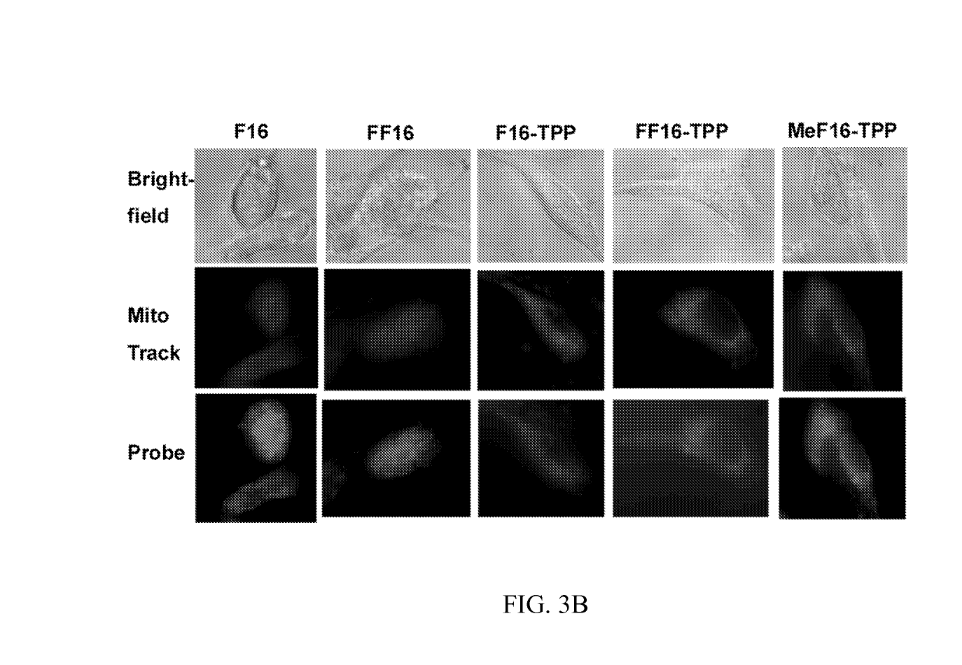

A theranostic agent that combines diagnosis and therapy simultaneously would be of great significance for clinical applications. Considering the specific mitochondria targeting ability of both TPP and F16, as well as the imaging ability and cytotoxicity toward various cancer cell lines of F16, we proposed that coupling F16 analogues with TPP might provide novel agents for cancer imaging and treatment. Such potential theranostic agents would have the following advantages. First, TPP is a molecule which cannot be imaged directly. Tedious and costly procedures are required to include radionuclides such as ¹⁸F for visualization of diseased tissue (Min et al. (2004) J. Nucl. Med. 45:636-643; Cheng et al. (2005) J. Nucl. Med. 46:878-886; Gurm et al. (2012) JACC Cardiovasc Imaging 5:285-292; Kim et al. (2012) Bioconjug. Chem. 23:431-437; Madar et al. (2007) Eur. J. Nucl. Med. Mol. Imaging 34:205720-205765; Wang et al. (2007) J. Med. Chem. 50:5057-5069; Kim et al. (2008) J. Med. Chem. 51:2971-2984; Chalmers et al. (2012) J. Am. Chem. Soc. 134:758-761; Cocheme et al. (2012) Nat. Protoc. 7:946-958). Coupling TPP with F16 could thus provide TPP analogues with optical imaging and cytotoxic ability simultaneously. Such conjugates would be useful in various applications, such as cell mitochondria imaging and image guided surgery, which needs good fluorescence contrast between cancer and normal tissues (Keereweer et al. (2011) Mol. Imaging Biol. 13:199-207). Second, since both F16 and TPP analogues possess mitochondrial targeting ability, coupling them together results in DCLs with two positive charges which can likely maintain the mitochondria targeting ability. The functions of F16-TPP analogues for mitochondria targeting are shown in FIG. 3.

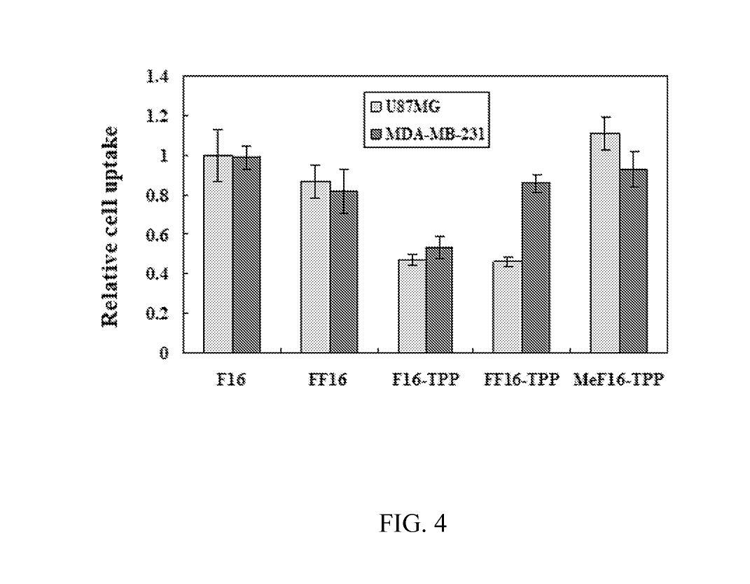

In this study, three F16-TPP analogues (F16-TPP, FF16-TPP and MeF16-TPP) bearing different substituents were synthesized along with F16 and an F16 derivative (FF16) for comparison. The conjugates were synthesized in a way similar to F16 as shown in FIG. 4. The intermediate 6 was prepared by reacting 4-picoline and (4-bromobutyl)triphenylphosphonium bromide readily in ethyl acetate at a good yield of 72.4%. The following condensation of 6 and indole-3-carboxaldehyde analogues in methanol and purification with reverse HPLC thereafter gave the TPP-F16 derivatives yet at low yields (<15%).

Optical properties of all F16 related compounds were studied at a concentration of 5 μM in PBS buffer. As shown in the spectra of the compounds (FIG. 3A), all the compounds exhibited similar characteristics with close absorption and emission wavelengths for each other. The maximum absorption and emission are around 425 nm and 525 nm, respectively. Since TPP moiety is non-fluorescent, it is expected that F16-TPP conjugates inherit F16's optical characteristics. However, subtle influences of substituents on F16 and TPP moiety could be observed. For example, as shown in FIG. 4A, both FF16-TPP and MeF16-TPP exhibited increasing fluorescence intensity at different levels in contrast to F16. Moreover, MeF16-TPP showed significant red shift of their absorption and emission wavelengths compared to F16.

Tumor cell uptake of all F16 related compounds were studied by incubating two cancer cell lines (U87MG and MDA-MB-231) with the probes for 1 hour and then imaging was performed with a fluorescent microscope. Meanwhile, to investigate the localization of the F16 derivatives, after incubation with the probes, cells were co-stained with the commercially available dye Mitotrack, which is widely used for mitochondria staining. The images of F16 related compounds in U87MG cells were displayed in FIG. 3B. Under eGFP filter set (ξex 450/490 nm, λem 515/565 nm), no significant autofluorescence of the cells were observed, making it convenient to study the relative uptake of the probes directly by comparing their fluorescence signals. Importantly, all compounds showed specific accumulations in the mitochondria of the tumor cells, which was proven by good overlay of images under the condition of the co-staining of the probes and Mitotrack. These results demonstrated the mitochondria targeting ability of F16 analogues and F16-TPP conjugates.

We then investigated the uptake of all the probes in U87MG and MDA-MB-231 cells at 1 hour incubation by quantitative analysis of fluorescent signals intensity of all F16 related compounds in the cells. The cellular uptake experiments were repeated nine times, and the relative cellular uptakes ability of different probes were calculated by comparing their percentage of cellular fluorescent signals and then normalized by that of F16 (FIG. 4). For these probes, their uptake performance in U87MG and MDA-MB-231 was in general quite similar. For instance, F16, FF16, F16-TPP and MeF16-TPP all showed similar uptakes in both cell lines (P>0.05). Notably, only FF16-TPP displayed a dramatically increased uptake in MDA-MB-231 cells than that in U87MG (P<0.05). FIG. 4 also revealed the structural impact of the F16 related compounds towards their uptakes. For U87MG cells, F16, FF16 and MeF16-TPP all showed high and comparable uptakes (˜1), while F16-TPP and FF16-TPP exhibited much lower uptakes than other probes (approximately half of the uptake of F16, P<0.05). For MDA-MB-231 cell line, all F16 derivatives showed comparable uptakes except F16-TPP with about half uptake in contrast to the other probes.

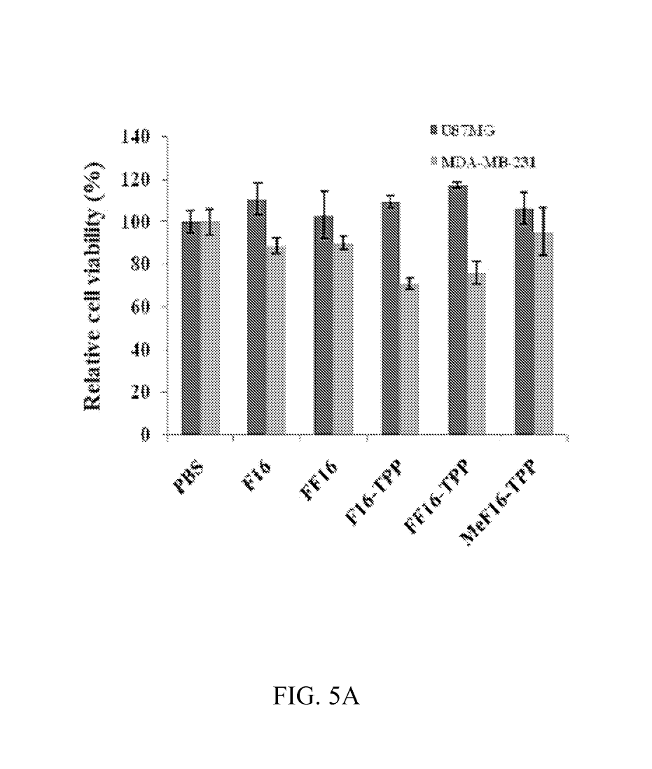

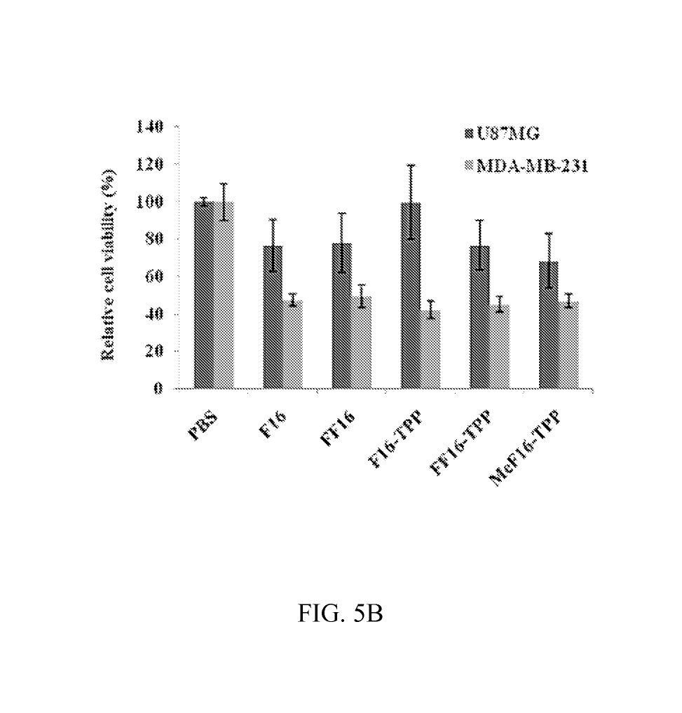

Apart from the imaging studies, we then investigated anti-tumor activities of all F16 related compounds by studying their antiproliferative effects in U87MG and MDA-MB-231 cell lines. After exposure to 5 or 10 μM of the compounds in the cancer cells for 4 days, the cell proliferative ratios to the control were measured (see FIG. 5). At a lower concentration of 5 μM, all compounds exhibited no antiproliferative activities for U87MG cell line, while low to moderate activities with an antiproliferative ratio less than 30% could be observed for the MDA-MB-231 cell line (FIG. 5A). With increasing concentration, the compounds showed distinct antitumor activities in two cell lines. As shown in FIG. 5B, at a concentration of 10 μM, all the compounds prepared displayed weak antitumor activities as indicated by their antiproliferative ratios of less than 32% for the U87MG cell line. However, for the MDA-MB-231 cell line, all compounds showed much stronger cytotoxicities with similar antiproliferative ratios of over 50%. The different antiproliferative activities imply that the antitumor potency of the F16 related compounds is cell-dependent. Meanwhile, substituents in F16 and F16-TPP analogues played distinct roles, for instance, introduction of a fluorine atom in F16 barely influences activity of FF16 (P>0.05), while substitution of fluorine and methyl groups apparently impacts F16-TPP analogues in the U87MG cell line at a higher concentration of 10 μM.

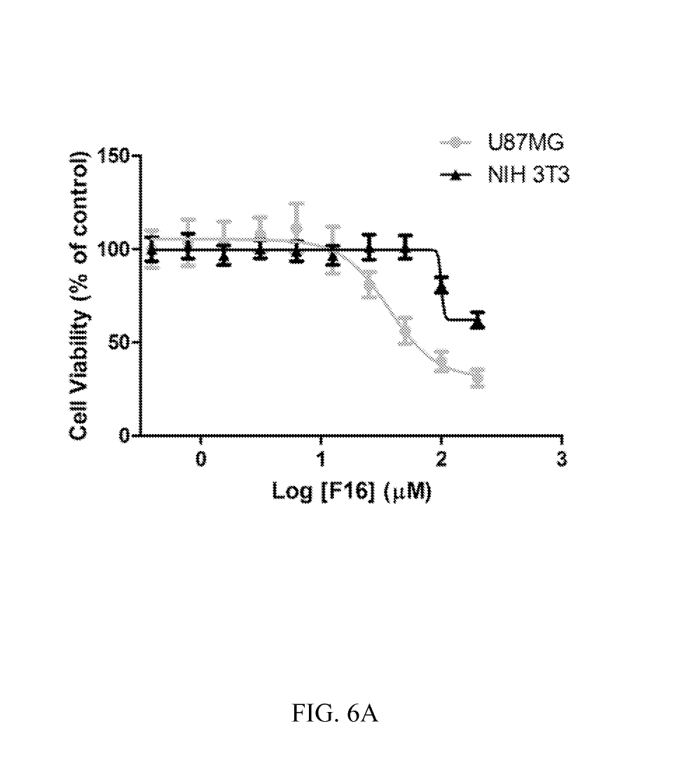

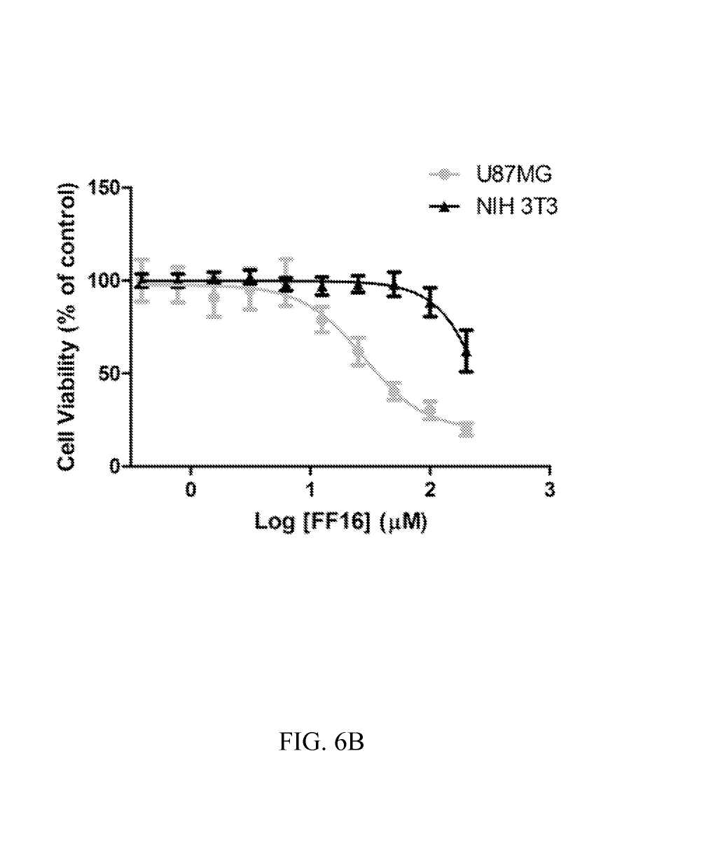

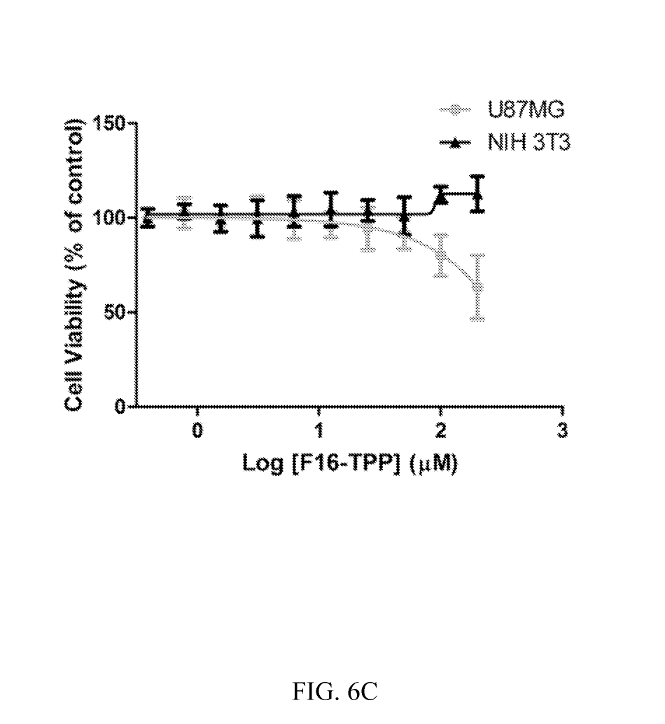

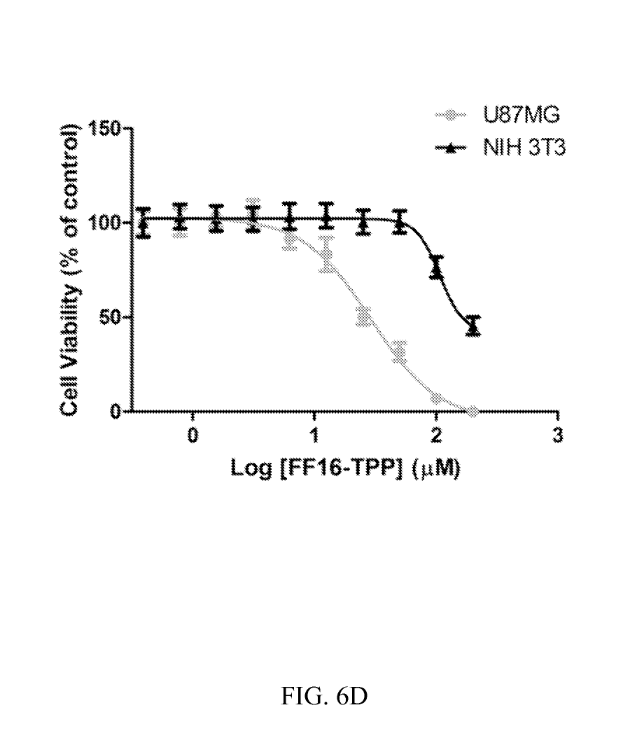

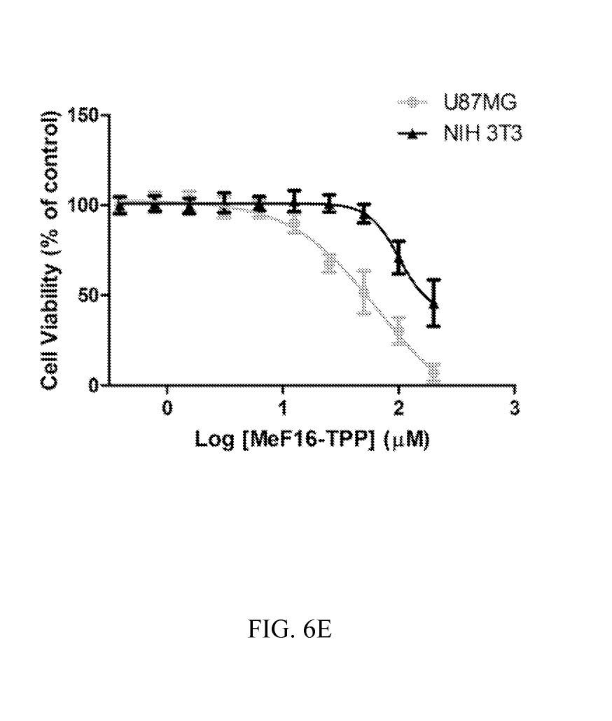

To further confirm the relationship between cell uptake and antitumor activity, the half inhibitory concentrations (IC₅₀) of these compounds against the U87MG cell line were measured. The IC₅₀ for F16, FF16, F16-TPP, FF16-TPP and MeF16-TPP are 36.5±1.1, 28.0±1.2, >200, 28.9±1.1, and 64.0±1.3 μM, respectively (Table 1, FIG. 6). Interestingly, substitution of fluorine in the F16 molecule slightly improves the bioactivity of the resulting compound (˜1.3 fold), whereas adding a fluorine in F16-TPP dramatically improves the bioactivity of the resulting compound (>6.9 fold). Moreover, adding a methyl group to F16-TPP also improved its toxicity over 3 fold. It should be noted that all these five compounds show minimum or even un-observable toxicity in the fibroblast cell line NIH 3T3 (IC₅₀ all >100 μM, Table 1, FIG. 6), highlighting the treatment specificity of these mitochondrial targeted agents.

TABLE 1
Summary of the half inhibitory concentration (IC₅₀) of various
compounds against both U87MG cells and NIH 3T3 cells.

IC50 (μM)*

| Samples | U87MG | NIH 3T3 |
|---|---|---|
| F16 | 36.5 ± 1.1 | ~100 |
| FF16 | 28.0 ± 1.2 | ~491 |
| F16-TPP | >200 | N.O.T. ^{†} |
| FF16-TPP | 28.9 ± 1.1 | ~111.1 |
| MeF16-TPP | 64.0 ± 1.3 | ~109.6 |

*Both cell lines at a density of 3000 cells/well were incubated in 96-well plates for 4 days. The viable cell numbers were checked and directly counted under microscopy (10X). A minimum of 1 mm × 1 mm area was counted from each of at least three widely separated regions of cell culture.
^{†} N.O.T.: Not obvious toxicity.

Our cell imaging and treatment study confirmed that F16-TPP analogues preserve the tumor cell mitochondria targeting ability and can be used for cancer cell fluorescence imaging and treatment. Especially, we successfully developed a theranostics agent FF16-TPP, which showed fluorescence imaging ability and increased activity compared to F16. Meanwhile, we discovered that FF16 also showed superior cell killing ability compared to F16, demonstrating a simple fluorination of F16 can improve its anti-tumor activity while maintaining its optical properties.

One thing to note is that F16-TPP shows low cell uptake and killing ability, which is beyond our initial expectation for synergistic effects that the conjugate should bring about. This may be ascribed to the fact that the F16-TPPs bear more positive charges than F16 (2+ versus 1+), which may lead to the reduced permeability of the conjugates into cells. Further substituting a lipophilic methyl group or electronegative fluorine atom in F16-TPP, MeF16-TPP and FF16-TPP greatly enhanced cell killing capability (FIG. 4, Table 1 and FIG. 6). These data suggest the importance of fine tuning the structure of F16-TPP to achieve high cancer cell killing ability.

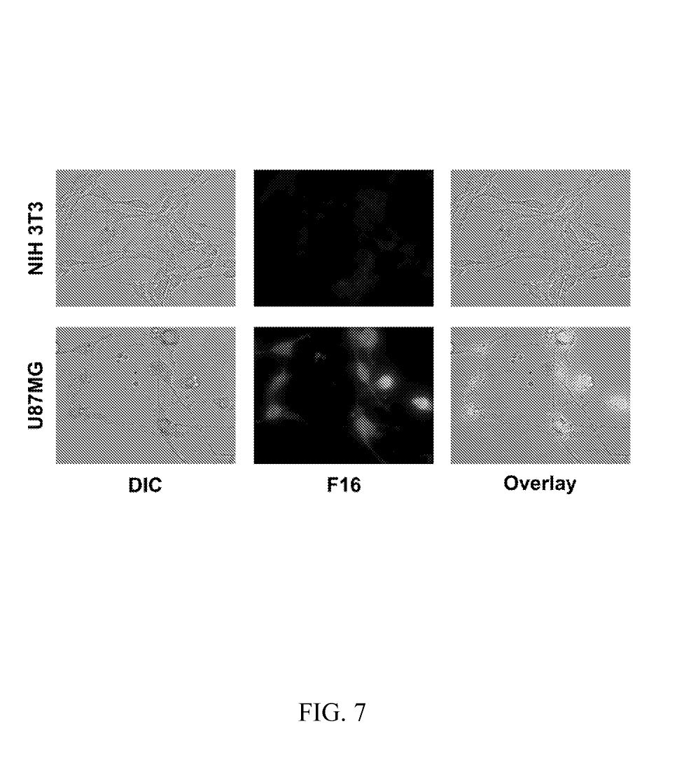

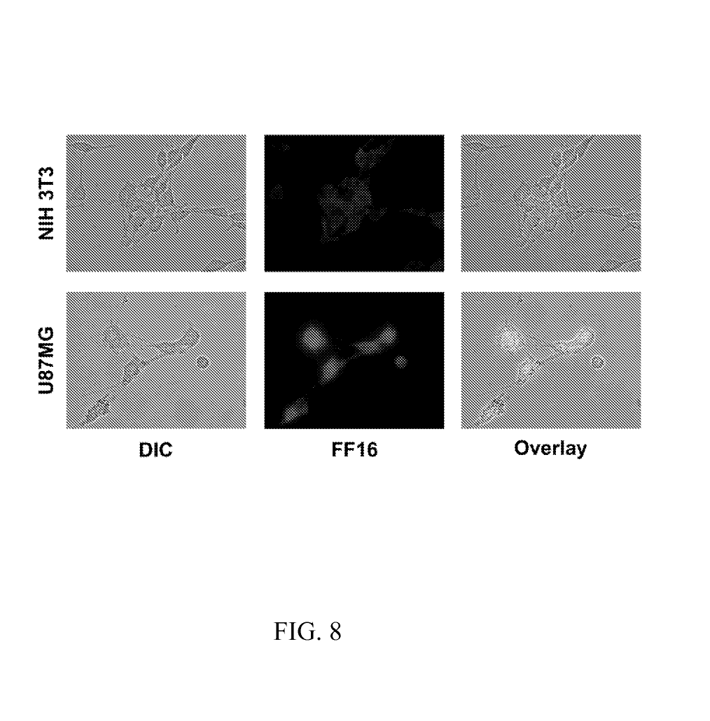

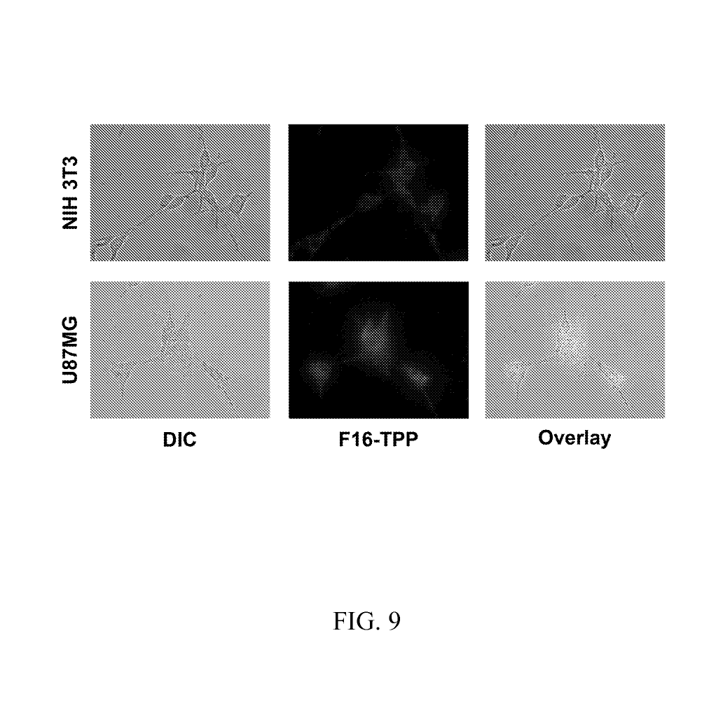

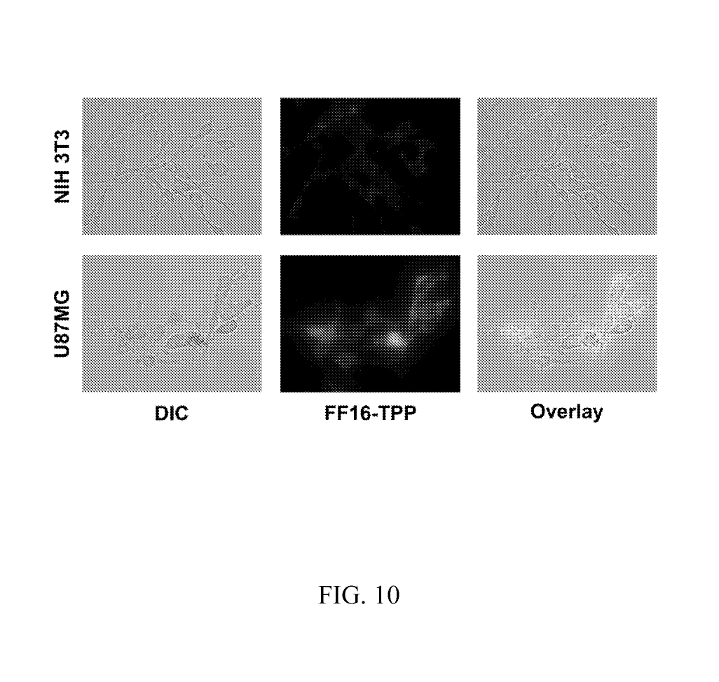

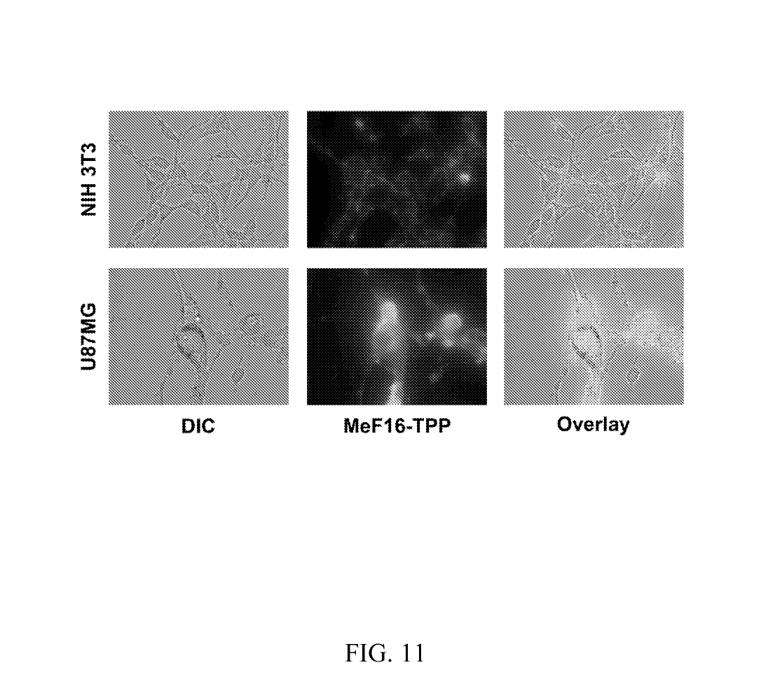

Since TPP itself does not impart cytotoxicity as revealed by some studies (Millard et al. (2010) Plos One 5:e13131), it is reasonable to hypothesize that F16-TPP conjugates kill tumor cells in a similar way as F16 by higher accumulation in tumor cells than in normal cells, which is caused by higher membrane potentials of mitochondria (ΔΨm) in tumor cells (Fantin et al. (2002) Cancer Cell 2:29-42; Fantin et al. (2004) Cancer Res. 64:329-336). Indeed F16 and F16-TPP analogues all displayed much higher accumulations in U87MG cells than that in NIH 3T3 cells (P<0.05, FIGS. 7-11). Apparently, this cytotoxicity is associated with accumulation level of the compounds. This may explain why F16 related compounds show higher anti-tumor activities at higher concentration of 10 μM than those at 5 μM. As for distinct cytotoxities of the same compound in different cell lines, these may be attributed to the distinct membrane potentials of mitochondria between the two cell lines we used. However, substituents like fluorine and methyl group exhibit negligible impacts on cytotoxities to MDA-MB-231 cell line at 10 μM, which are quite different from the uptake tendency shown in FIG. 4. This may be caused by different time courses used for two studies (1 hour for cell uptake assay and 4 days for proliferation assay). Another possibility is that mitochondria accumulation may not be the only factor that influences the antiproliferative activities. Different total charges and charge distributions may also affect their capability on reducing ΔΨm to result in further biological cascade effects such as inhibition of mitochondria respiration and cell death.

### CONCLUSIONS

The fluorescent mitochondria-specific agents, F16 analogues and F16-TPP conjugates were successfully synthesized. Especially, FF16 and FF16-TPP showed higher potency and comparable or increased mitochondria accumulation in tumor cell lines compared to F16, making them excellent candidates for mitochondria targeted optical imaging and treatment. Moreover, the structural modification of F16 related compounds showed high impacts on their cell uptake and antitumor activities. Our findings will not only benefit development of mitochondria-targeted theranostic agents based on TPP and F16, but also expand the usage of TPP as a mitochondria carrier.

Materials and Methods

General.

All chemicals were purchased from Sigma-Aldrich Chemical Co. (St. Louis, Mo.) and used without further refinement. Purification and analysis of the F16-TPP analogues were performed with the Dionex Summit high-performance liquid chromatography (HPLC) system (Dionex Corporation, Sunnyvale, Calif.), equipped with a 340U four-channel UV-Vis absorbance detector. Reverse-phase HPLC column Dionex Acclaim 120 (C18, 4.6 mm×250 mm) was used for analysis of the products, while reverse-phase semi-preparative HPLC column Zorbax SB (C18, 9.4 mm×250 mm) was used for purification of the products. The mobile phase was 0.1% trifluoroacetic acid (TFA) and 0.1% TFA in acetonitrile (CH₃CN). The flow were 1 mL/minute for analysis and 4 mL/minute for separation with the gradient starting from at 5% CH₃CN and ending at 65% CH₃CN at 42 minutes. UV wavelengths used for detection of all F16 derivatives were 218 nm and 440 nm. Electron spray ionization (ESI) mass spectrometry was performed by Vincent Coats Foundation Mass Spectrometry Laboratory, Stanford University. All NMR spectra were performed on a Varian XL-400 (Varain, Palo Alto, Calif.).

### Synthesis of (E)-4-(1H-indol-3-ylvinyl)-N-methylpyridinium iodide (F16, compound 4)

F16 was prepared according to the procedure that reported with minor modification (Wang et al. (2001) Acta. Cryst. Section C, 57(Pt 11):1343-1348; herein incorporated by reference). Briefly, equivalent mole of 1,4-dimethylpyridium iodide (1 mmol) with indole-3-carboxaldehyde (1 mmol) in the presence of catalytic amount of piperidine in 10 mL of methanol was refluxed for 5 hours with continuous stirring. The precipitate was collected, washed with methanol and recrystallized with acetonitrile to give the product as orange powder (yield: 56.0%). ¹H NMR (D₂O, 400 MHz): δ(ppm) 8.48 (d, J=8.0 Hz, 2H), 8.21 (d, J=16.0 Hz, 1H), 8.07 (m, 1H), 8.00 (d, J=8.0 Hz, 2H), 7.84 (s, 1H), 7.47 (m, 1H), 7.26 (m, 2H), 7.24 (d, J=16.0 Hz, 1H), 4.20 (s, 3H).

### Synthesis of (E)-4-(1H-5-fluro-indole-3-ylvinyl)-N-methylpyridinium iodide (FF16, compound 5)

Preparation of compound 5 was conducted with the procedure similar to F16 at a yield of 52.2%. ¹H NMR (D₂O, 400 MHz): δ(ppm) 8.51 (d, J=8.0 Hz, 2H), 8.14 (d, J=16.0 Hz, 1H), 8.04 (d, J=8.0 Hz, 2H), 7.89 (s, 1H), 7.78 (d, J=8.0 Hz, 1H), 7.43 (d, J=8.0 Hz, 1H), 7.20 (d, J=16.0 Hz, 1H), 7.04 (dd, J=8.0 Hz, 1H), 4.22 (s, 3H). MS(ESI+): 253.2 (for calculated C₁₆H₁₄FN²⁺ 253.3).

### Synthesis of 4-picoline-TPP 6

4-picoline (1 mL, 10.2 mmol) and (4-bromobutyl)triphenylphosphonium bromide (4.88 g, 10.2 mmol) were dissolved in 50 mL of ethyl acetate. The solution was stirred at room temperature for 2 days before the solvent was removed under vacuum. The residue was then washed with dichloromethane (3×30 mL) and dried to give the product as pale powder. Yield: 72.4%. ¹H NMR (DMSO-d₆, 400 MHz): δ(ppm) 8.87 (d, J=8.0 Hz, 2H), 7.95 (d, J=8.0 Hz, 2H), 7.89 (m, 3H), 7.77 (m, 12H), 4.54 (t, J=8.0 Hz, 2H), 3.64 (s, 3H), 2.58 (m, 2H), 2.07 (m, 2H), 1.50 (m, 2H). MS(ESI+): 205.8 (for calculated C₂₈H₃₀NP²⁺/2 205.8).

General Procedures for Synthesis of F16-TPP Derivatives.

Compound 6 and equivalent amount of indole-3-carboxaldehyde analogue was dissolved in methanol and refluxed overnight in the presence of catalytic amount of piperidine (5% molar ratio). The dark red residue obtained by evaporation of the solvent of the mixture was purified by HPLC on a semipreparative C-18 column. The flow rate was set as 3 mL/minute, with the mobile phase starting from 95% solvent A and 5% solvent B (0-3 minutes) to 35% solvent A and 65% solvent B at 33 minutes, then going to 15% solvent A and 85% solvent B (33-36 minutes), maintaining this solvent composition for another 3 minutes (36-39 minutes), and going back to the initial composition by 42 minutes. The desired fractions were collected, concentrated, and lyophilized to give the product as orange to dark red powder.

F16-TPP: ¹H NMR (D₂O, 400 MHz): δ(ppm) 8.00 (d, J=8.0 Hz, 2H), 7.91 (m, 1H), 7.85 (d, J=16.0 Hz, 1H), 7.71 (s, 1H), 7.64 (m, 2H), 7.54-7.40 (m, 15H), 7.39 (m, 1H), 7.17 (m, 2H), 6.75 (d, J=16.0 Hz, 1H), 4.14 (t, 2H), 3.12 (m, 2H), 1.94 (m, 2H), 1.35 (m, 2H). MS(ESI+): 269.3 (for calculated C₃₇H₃₅N₂P²⁺/2 269.3).

FF16-TPP: ¹H NMR (D₂O, 400 MHz): δ(ppm) 8.01 (m, 1H), 7.90 (d, J=8.0 Hz, 2H), 7.63 (d, J=16.0 Hz, 1H), 7.59 (s, 1H), 7.46-7.37 (m, 15H), 7.32 (d, J=8.0 Hz, 2H), 7.22 (m, 1H), 7.01 (m, 1H), 6.80 (m, 1H), 6.58 (d, J=16.0 Hz, 1H), 4.08 (t, J=8.0 Hz, 2H), 3.09 (m, 2H), 1.90 (m, 2H), 1.33 (m, 2H). MS(ESI+): 279.0 (for calculated C₃₇H₃₄FN₂P²⁺/2 278.4).

MeF16-TPP: ¹H NMR (D₂O, 400 MHz): δ(ppm) 8.47 (d, J=8.0 Hz, 2H), 8.16 (d, J=16.0 Hz, 1H), 7.81 (m, 4H), 7.74 (m, 12H), 7.36 (d, J=8.0 Hz, 1H), 7.22 (d, J=16.0 Hz, 1H), 7.12 (d, J=8.0 Hz, 1H), 4.43 (t, J=8.0 Hz, 2H), 3.48 (m, 2H), 2.52 (s, 3H), 2.06 (m, 2H), 1.75 (m, 2H). MS (ESI+): 276.4 (for calculated C₃₈H₃₇N₂P²⁺/2 276.3).

Cell Lines.

Human breast cancer cell line MDA-MB-231, glioma cell line U87MG, and mouse embryonic fibroblasts cell line NIH 3T3 were obtained from American Type Culture Collection (Manassas, Va.). U87MG cells were cultured in minimum essential medium (Eagle) (MEM, Invitrogen, Carlsbad, Calif.) supplemented with 0.1 mM nonessential amino acids, 1 mM sodium pyruvate, 1.5 g/L sodium bicarbonate, 0.01 mg/mL bovine insulin, 10% fetal bovine serum (FBS), and 1% penicillin-streptomycin. MDA-MB-231 and NIH 3T3 cells were grown in Dulbecco's modified Eagle high glucose medium (DMEM, Invitrogen, Carlsbad, Calif.) supplemented with 10% FBS and 1% penicillin-streptomycin.

Fluorescence Microscopy Studies.

U87MG or MDA-MB-231 cell lines (1×10⁵) were incubated in 35 mm MatTek glass-bottom cultures dishes (Ashland, Mass.). The cells were washed with PBS (pH=7.4) after 24 hours incubation and then incubated with the compounds separately for a further 1 hour. Thereafter cells were washed three times with ice-cold PBS. The fluorescent signal of the cells was measured with an Axiovert 2000M fluorescence microscope (Carl Zeiss Micro-Imaging, Inc., Thornwood, N.Y.) with the eGFP filter set (excitation 450/490 nm, emission 515/565 nm). An AttoArc HBO 100 W microscopic illuminator was used as a light source. Images were recorded with a thermoeletrically cooled charged-coupled device (CCD) (Micromax, model RTE/CCD-576, Princeton Instruments, Inc., Trenton, N.J.) and analyzed with MetaMorph software version 6.2r4 (Molecular Devices Corporation, Downingtown, Pa.).

Cell Proliferation Assay.

Both U87MG and NIH 3T3 cell lines at a density of 3000 cells/well were incubated in 96-well plates overnight. The culture medium was replaced with 200 μL of culture medium in which the testing compound dispersed at various concentrations (0.5-200 μM). After incubation for 4 days, the viable cell numbers were checked and directly counted under microscopy (10×). A minimum of 1 mm×1 mm area was counted from each of at least three widely separated regions of cell culture. The cell proliferation rate was calculated by the following formula: cell proliferation rate (%)=(average cell number of sample wells/average cell number of control wells×100. The intact culture medium was evaluated as a control.

Statistical Methods.

All data were presented as mean±SD. Means were compared using the Student's t-test. A 95% confidence level was chosen to determine the significance between groups, with P values of <0.05 indicating statistically significant differences.

While the preferred embodiments of the invention have been illustrated and described, it will be appreciated that various changes can be made therein without departing from the spirit and scope of the invention.

What is claimed is:

1. A compound having the formula: 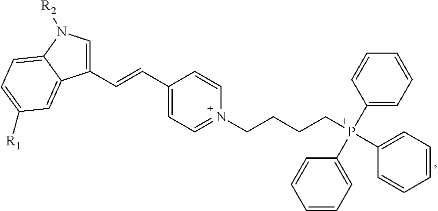
or a pharmaceutically acceptable salt thereof, wherein R₁ is a hydrogen atom, a halogen atom, an alkyl group, or an aryl group, and R₂ is a hydrogen atom or an alkyl group.

2. The compound of claim 1 having the formula: 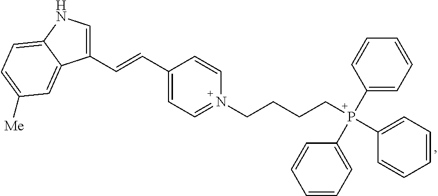
or a pharmaceutically acceptable salt thereof.

3. The compound of claim 1 having the formula: 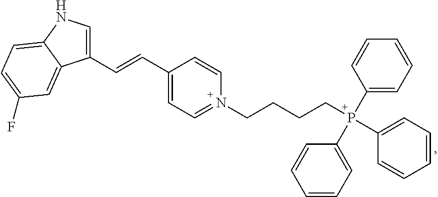
or a pharmaceutically acceptable salt thereof.

4. The compound of claim 1 having fluorescence, cytotoxicity, and mitochondrial targeting characteristics.

5. The compound of claim 1 having the ability to cause apoptosis and decrease cell proliferation of a target cell.

6. The compound of claim 1, wherein the compound is selectively cytotoxic to cancer cells.

7. The compound of claim 1, wherein the compound can be used for in vivo imaging of cells that uptake the compound into mitochondria.

8. The compound of claim 1, wherein R₁ is a halogen selected from the group consisting of fluorine, chlorine, and bromine.

9. The compound of claim 1, wherein R₂ is a hydrogen atom or a methyl group.

10. A composition comprising the compound of claim 1 and a pharmaceutically acceptable excipient.

11. The composition of claim 10, further comprising a chemotherapeutic agent.

12. A method for treating a subject for a disease or disorder associated with mitochondrial dysfunction, the method comprising administering to the subject a therapeutically effective amount of the composition of claim 10, wherein the compound causes apoptosis and decreases cell proliferation of target cells in the subject that uptake the compound into mitochondria.

13. The method of claim 12, further comprising monitoring uptake of the compound by mitochondria in cells of the subject by detecting fluorescence from the compound.

14. The method of claim 12, further comprising recording a fluorescence image of cells that uptake the compound into mitochondria of the subject.

15. A method for treating cancer comprising administering to a subject in need thereof a therapeutically effective amount of the composition of claim 10.

16. The method of claim 15, further comprising monitoring uptake of the compound by mitochondria in cells of the subject by detecting fluorescence from the compound.

17. The method of claim 15, further comprising recording a fluorescence image of cells that uptake the compound into mitochondria of the subject.

18. The method of claim 17, wherein the cells are cancerous cells or cells of a tumor.

19. The method of claim 18, further comprising monitoring anti-tumor activity of the compound by recording one or more fluorescence images of cells after uptake of the compound into mitochondria of the subject.

20. The method of claim 19, wherein one or more fluorescence images are recorded with a medical fluorescence imaging system.

21. The method of claim 20, wherein the medical fluorescence imaging system is a handheld fluorescence microscope, laparoscope, endoscope, or microendoscope.

22. The method of claim 15, further comprising administering a therapeutically effective amount of a chemotherapeutic agent.

23. The method of claim 15, wherein the cancer is breast cancer or glioma.

24. The method of claim 15, wherein multiple cycles of treatment are administered to the subject for a time period sufficient to effect at least a partial tumor response.

25. The method of claim 24, wherein multiple cycles of treatment are administered to the subject for a time period sufficient to effect a complete tumor response.

26. A method of making the compound of claim 1, the method comprising:
a) reacting a 1,4-dimethylpyridinium salt in the presence of catalytic amounts of piperidine with an indole compound having the formula: 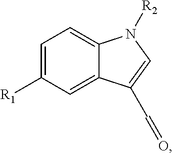
wherein R₁ is a hydrogen atom, a halogen atom, an alkyl group, or an aryl group, and R₂ is a hydrogen atom or an alkyl group, to produce a first reaction intermediate;
b) reacting 4-picoline with a (4-bromobutyl)triphenylphosphonium salt to produce 4-picoline-alkyltriphenylphosphonium as the second reaction intermediate; and
c) reacting the first reaction intermediate with the second reaction intermediate in the presence of catalytic amounts of piperidine to produce the compound of claim 1.

27. The method of claim 26, wherein the indole compound is selected from the group consisting of indole-3-carboxaldehyde, 5-fluro-indole-3-carboxaldehyde, and 5-methyl-indole-3-carboxaldehyde.

28. A method of using the compound of claim 1 for monitoring mitochondria in a cell, the method comprising:
a) contacting the cell with the compound of claim 1, wherein mitochondria of the cell uptake the compound;
b) illuminating the cell with light at a fluorescence excitation wavelength of the compound; and
c) detecting fluorescence emitted by the compound.

29. The method of claim 28, wherein fluorescence is detected by a fluorimeter, a fluorescence microscope, a fiber-optic fluorescence imaging system, a fluorescence microplate reader, a fluorometric imaging plate reader, fluorescence-activated cell sorting, or a medical fluorescence imaging device.

30. A method of using the compound of claim 1 for fluorescence imaging of a cell, the method comprising:
a) contacting the cell with the compound of claim 1, wherein mitochondria of the cell uptake the compound;
b) illuminating the cell with light at a fluorescence excitation wavelength of the compound; and
c) recording a fluorescence image of the cell by detecting fluorescence emitted by the compound.

31. The method of claim 30, wherein a fluorescence image is visualized with a fluorescence microscope, a fiber-optic fluorescence imaging system, or a medical fluorescence imaging device.

32. A method of simultaneously treating and imaging a tumor, the method comprising:
a) contacting the tumor with the compound of claim 1, wherein mitochondria in cells of the tumor uptake the compound, thereby causing apoptosis and decreasing cell proliferation of the cells of the tumor;
b) illuminating the tumor with light at a fluorescence excitation wavelength of the compound; and
c) detecting fluorescence emitted by the compound from mitochondria in the cells of the tumor.

33. A method of performing fluorescence image-guided surgery on a subject, the method comprising:
a) contacting mitochondria in a tissue of interest with the compound of claim 1, wherein the mitochondria uptake the compound;
b) illuminating the tissue of interest with light at a fluorescence excitation wavelength of the compound;
c) recording a fluorescence image by detecting fluorescence emitted by the compound with a fluorescence imaging system; and
d) performing surgery on the subject.

34. The method of claim 33, wherein the fluorescence imaging system comprises a handheld fluorescence microscope, laparoscope, endoscope, or microendoscope.

35. The method of claim 33, wherein fluorescence imaging is used for detection of pathology, evaluation of the completeness of resection, visualization of critical structures, or evaluation of the efficacy of treatment.

36. The method of claim 33, wherein a fluorescence image is recorded by a charge-coupled device (CCD) image sensor, a CMOS image sensor, or a digital camera.

37. A kit comprising the compound of claim 1.
